(19) United States
(12) Patent Application Publication
(10) Pub. No.: US 2007/0087353 A1
(43) Pub. Date: Apr. 19, 2007

(54) Microarray biochemical reaction device

(75) Inventors: Ji-Yen Cheng, Taipei (TW); Cheng-Wey Wei, Taipei (TW)

Correspondence Address: BACON & THOMAS, PLLC, 625 SLATERS LANE, FOURTH FLOOR, ALEXANDRIA, VA, 22314, US

(73) Assignee: Academia Sinica, Taipei (TW)

(21) Appl. No.: 11/249,449

(22) Filed: Oct. 14, 2005

(51) Int. Cl. C12Q 1/68; C12M 3/00

(52) U.S. Cl. 435/6; 435/287.2

(57) ABSTRACT

The microarray biochemical reaction device of this invention integrates microfluidic trenches with a microarray to form a serpentine microchannel passing through all DNA probes provided in the microarray. A sample solution is introduced into the microchannel and scrambled into discrete plugs to induce droplet mixing. The discrete plugs are then shuttled through the entire microchannel (shuttle hybridization), sweeping over DNA probes to perform hybridization. Using chaotic mixing of droplets, the hybridization efficiency is enhanced and reaction time for hybridization is shortened. During shuttling, the plugs are thoroughly mixed by the natural re-circulating flows. Method for the preparation of the microarray biochemical reaction device is also disclosed.

### FIELD OF THE INVENTION

The present invention relates to a microarray biochemical reaction device, especially to a biochemical reaction device combining a microarray and a microchannel.

### BACKGROUND OF THE INVENTION

The DNA microarray is a highly effective approach for high-throughput gene expression analysis and genotyping. Target DNA molecules suspended in the solution can pair with surface-bound DNA probes through heterogeneous DNA-DNA hybridization to determine simultaneously the relative concentration of multiple targets in the sample.

Microarray analysis has become a powerful technology for drug screening, disease gene identification and signaling pathway studies. However, one of the serious limitations on the reaction of biomolecules is the slow diffusion kinetics. For instance, in a DNA-DNA hybridization experiment, the pairing reaction normally takes more than 12 hours (over night) to run to completion. Furthermore, the sample and reagent volumes required for conventional DNA-DNA hybridization reaction are quite large. A 100 μl sample is usually consumed for a 25×75 mm slide that bears 10,000 probes. The large amount required sometimes constrains the practical application of the powerful DNA microarray.

Thermodynamic equilibrium is critical in heterogeneous DNA hybridization assay. Considerable cross hybridization occurs under non-equilibrium conditions. Furthermore, a lower target concentration corresponds to a longer equilibrium time. Genes that are down-regulated require longer times to be measured with the same accuracy as those that are up-regulated. For optimized probes for which cross-hybridization is very low, the time taken to reach equilibrium still depends on target concentration. Therefore, systematic hybridization bias is frequently found when the hybridization reaction is not driven to completion.

The diffusion constant of DNA in water is extremely low (Dw=4.9×10⁻⁹ cm²/s×[bp]^{-0.72}, Dw=2.1×10⁻⁷ cm²/s for 80-mer), so the equilibrium result is obtained after 12-15 hours at a concentration of 10⁻¹³-10⁻¹⁴ M (corresponding to 10⁶-10⁷ target copies per 100 μl of target solution). Hence, DNA microarray hybridization is typically performed overnight to ensure that the reaction runs to completion. Although in some hybridization stations convection motion is used to enhance hybridization, the effect is not significant. Some researchers have found that 66 hours is needed for complete hybridization so that the microarray result can be significantly improved.

Even when the reaction is performed overnight, not all of the target molecules can react with all of the surface-bound probes. For example, for 80-mer target DNA in water, the corresponding diffusion length (l=\sqrt{2Dt}, where D is the diffusion constant and t is the diffusion time) is 1.9 mm for one day. Restated, each probe has the opportunity to react only with nearby targets and the complementary targets that are outside the diffusion region are wasted. Normally, a 10,000-probe array is 20 mm×30 mm. The sample utilization efficiency is quite low under the diffusion process. Only ˜0.3% of the target in the sample is consumed.

In the conventional art, the DNA microarray experiment consumes ˜10 μg total RNA for a ˜10,000 probe microarray. The amount corresponds to about 2.5×10⁷ cells. The sample consumption can be greatly reduced by increasing the hybridization efficiency. If both the reaction time and the sample consumption are reduced, the application of the DNA microarray can be further expanded. For instance, low copy genes can be analyzed more accurately. Genotyping by microarray may be performed without amplifying the sample. Many strategies have been proposed to enhance the hybridization efficiency and reduce the hybridization time.

Cheek et al. employed a flow-through 3-D microchannel chip combined with chemiluminescence detection to reduce the signal variation and the detection limit. They achieved signal standard deviation of 8.1% and a detection limit of 250 attomoles. The hybridization time was 3 hour. The targets were 19 to 34 mer short oligonucleotides. See: Cheek, B. J. et al., (2001) “Chemiluminescence detection for hybridization assays on the flow-thru chip, a three-dimensional microchannel biochip”, Anal. Chem., 73, 5777-5783.

Adey et al. presented a unique microfluidic device that used pneumatically powered pumps for active mixing in a 25 μm-thick chamber to increase sensitivity by a factor of two to three. An air compressor generated a vacuum source and was used in the pneumatic system. A 35 μl sample was hybridized overnight in the device. The target was multiply labeled with Cy3 or Cy5 dCTP by PCR. A target quantity as low as 9.4 attomole was detected. See: Adey, N. B. et al. (2002) “Gains in sensitivity with a device that mixes microarray hybridization solution in a 25-mu m-thick chamber”, Anal. Chem., 74, 6413-6417.

Liu et al. used a special substrate surface combined with a microfluidic channel device to reduce the reaction time. The device exploited passive mixing by sample oscillation in a filled microfluidic channel to reduce the reaction time. 200 μl of 41 mer oligonucleotide is used in the hybridization. 1 fmol target was detected on the special surface in 15 minutes. See: Liu, Y. J. and Rauch, C. B. (2003) “DNA probe attachment on plastic surfaces and microfluidic hybridization array channel devices with sample oscillation”, Anal. Biochem., 317, 76-84.

Soper et al. used a PMMA microfluidic device assembled with a low-density array to reduce the hybridization time. In their study, four oligonucleotide probes (21-mer) with an inter-spot diameter of 400 μm were printed into a PMMA microfluidic channel. A hybridization signal with an S/N ratio of two was obtained with ten pM 21-mer targets labeled with infrared dye. See: Soper, S. A. et al. (2003) “Microarrays assembled in microfluidic chips fabricated from poly(methyl methacrylate) for the detection of low-abundant DNA mutations”, Anal. Chem., 75, 1130-1140.

Li et al. modeled the microarray hybridization kinetics of a microfluidic device. Reducing the channel height reduces the time required to reach a steady state. This effect is caused by the faster convection near the surface in the smaller channels. Hybridization in the microchannel indeed reduces the reaction time. A continuous flow of the hybridization solution is used in the modeling. See: Li, D. Q. et al. (2003) “Modeling of DNA hybridization kinetics for spatially resolved biochips”, Anal. Biochem., 317, 186-200.

Although reducing the dimensions of the microchannels reduces the diffusion length, a low Reynolds number for the microfluidic channel is known to cause a laminar flow that hinders effective mixing. The microchannel hybridization that employs the filled continuous sample flow involves this problem. Various measures have been developed to improve the mixing inside the channel.

Yaralioglu et al. applied on-chip ultrasonic transducers to cause active mixing around the transducers. The primary mixing mechanism is acoustic streaming. Mixing occurs near the region of the transducer. See: Yaralioglu, G. G. et al., (2004) “Ultrasonic mixing in microfluidic channels using integrated transducers”, Anal. Chem., 76, 3694-3698.

Stroock et al. demonstrated passive mixing by chaotic advection. The mixing is caused by chevron ridges embossed on the bottom of the channel. This method strategy has been demonstrated mainly for continuous liquid flow. See: Stroock, A. D. et al., (2002) “Chaotic mixer for microchannels”, Science, 295, 647-651.

### OBJECTIVES OF THE INVENTION

The objective of this invention is to provide a novel microarray reaction device, whereby reaction time for DNA hybridization may be shortened.

Another objective of this invention is to provide a novel microarray reaction device, whereby sample and reagent consumption for DNA hybridization may be reduced.

Another objective of this invention is to provide a novel microarray reaction device, whereby cycles of amplification in DNA hybridization may be reduced.

Another objective of this invention is to provide a novel microarray reaction device, whereby signal bias generated from DNA hybridization may be reduced.

Another objective of this invention is to provide a low cost microarray biochemical reaction device that is easy to prepare and convenient to use.

Another objective of this invention is to provide a method for the preparation of the above-said microarray biochemical reaction device.

### SUMMARY OF THE INVENTION

According to the present invention, a novel microarray biochemical reaction device is disclosed. The microarray biochemical reaction device of this invention integrates microfluidic trenches with a microarray to form a serpentine microchannel passing through all DNA probes provided in the microarray. A sample solution is introduced into the microchannel and scrambled into discrete plugs to induce droplet mixing. The discrete plugs are then shuttled through the entire microchannel (shuttle hybridization), sweeping over DNA probes to perform hybridization. Using chaotic mixing of droplets, the hybridization efficiency is enhanced and reaction time for hybridization is shortened. During shuttling, the plugs are thoroughly mixed by the natural re-circulating flows. The present invention provides a microarray biochemical reaction device with which a 1 μl target was used to hybridize with an array that can hold 5000 probes. This invention also discloses method for the preparation of the microarray biochemical reaction device.

These and other objectives of this invention may be clearly understood from the detailed description by referring to the following drawings.

### BRIEF DESCRIPTION OF THE DRAWINGS

FIG. 1 shows the structure of a microtrench prepared in a substrate.

FIG. 2 shows a microarray suited in the preparation of the microarray biochemical reaction device of this invention.

FIG. 3(a) shows the signal rise for various hybridization formats.

FIG. 3(b) presents details of the signal growth for a short hybridization time.

FIG. 3(c) presents the signal growth in shuttle hybridization with various channel depths.

FIG. 4 shows a histogram of the resultant fluorescence signal distribution obtained from hybridization using the present invention.

FIG. 5 presents the fluorescence images and the S/N ratio for various hybridization formats

### DETAILED DESCRIPTION OF THE INVENTION

Preparation of Microarray Biochemical Reaction Device

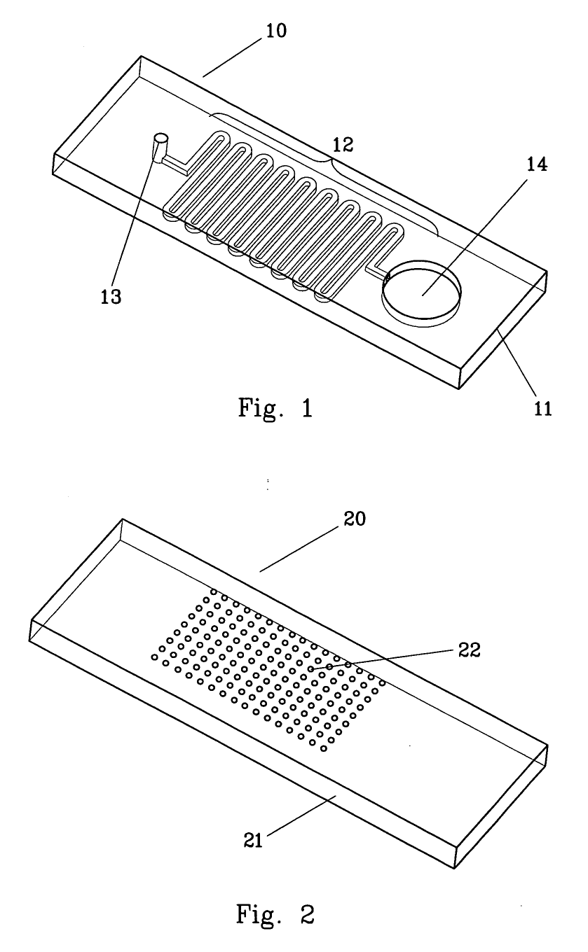

A commercial CO₂ laser scriber (M-300, Universal Laser Systems, USA) is used to engrave the PMMA substrate to fabricate a microtrench. The microtrench pattern is designed using CorelDraw (Corel) and then sent to the laser scriber for direct machining onto the PMMA substrate. FIG. 1 shows the structure of a microtrench prepared in a substrate according to this invention. As shown in this figure, 10 represents structure of the microtrench and 11 represents the substrate. In this embodiment, the substrate is a PMMA plate. Material of the substrate is not limited to PMMA. Other materials such as plastics, resin, glass, ceramics or other suited material may be used in this invention. 12 is a serpentine microtrench prepared in the substrate 11. The microtrench 12 has straight and parallel sections and bending sections, forming a continuous trench. Pattern of the microtrench 12 is of course not limited to any particular form. In some embodiments, the microtrench 12 may be polygonal, multiple arctic or spiral. The parallel sections may be straight or curved. The bending sections may be rectangular, in acute angles or obtuse angles, or round. The microtrench 12 may be prepared with a laser scriber, by dry or wet etching or any other method that produces accurate trench patterns. In addition, the microtrench is not limited to a continuous trench. It may have branches, as long as the microtrench may pass through all or most probes in the microarray after it is integrated with the microarray to be described hereinafter. The microtrench 12 is preferably provided with one inlet and one outlet or reservoir. The microtrench 12 as shown in this figure has one inlet 13 and one air chamber 14.

The trench width and depth can varied along with the laser fabrication parameters. Chips with trenches of depths 50 μm to 200 μm are preferable. Normally, a depth of 100 μm is used in hybridization, with a space of 100 μm to 200 separating them.

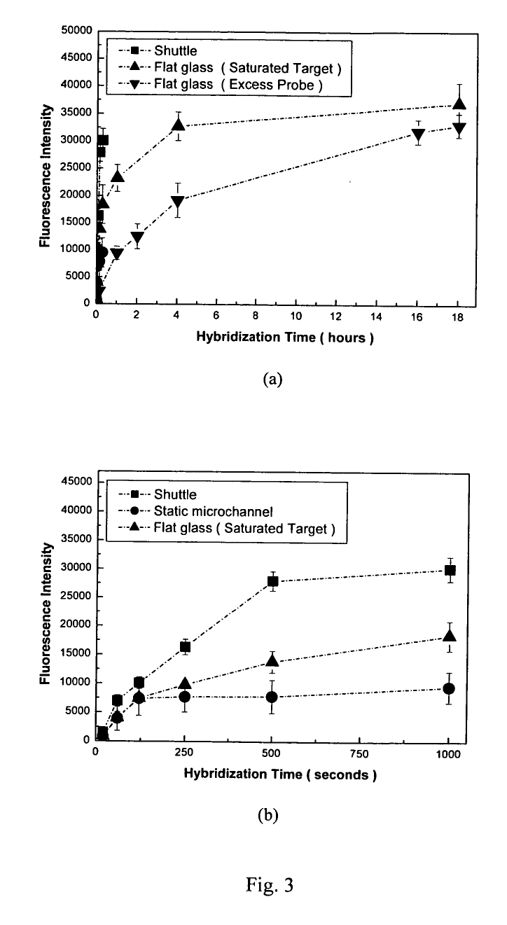

In forming a channel, the PMMA microtrench plate 10 is stacked with a conventional glass microarray. FIG. 2 shows a microarray suited in the preparation of the microarray biochemical reaction device of this invention. As shown in this figure, the microarray 20 is prepared in a glass substrate 21 and contains a plurality of probe spots 22. In this figure, 10×20, 200 probe spots are shown. However, this is not any limitation. Material of the substrate 21 is not limited to glass. Other materials such as plastics, resin and ceramics may also be used. If the substrate is glass, soda-lime glass, Pyrex glass, Borofloat glass or quartz (including crystal quartz and fused quartz) may be used. Basic requirements for the substrate are: high dimensional stability, low adsorption of molecules, and no contamination to the reaction system.

The two substrates 10 and 20 are stacked on top of each other, aiming the microtrench 12 to all probe spots in the microarray 21, a microarray with sealed microchannels is formed. The microchannel has only a single opening 13 to introduce a target solution and to connect to a syringe pump (not shown). Reagent solution is introduced and extracted from the opening 13. The sealed air chamber 14 at the distal terminal of the microchannel 12 is for storing compressed air.

The two plates 10 and 20 may be attached using ultrasonic welding, adhesives, or screws, or any applicable methods.

Hybridization

Probes are prepared in the probe spots 22 of the microarray 20. Oligonucleotide probes are dissolved in 0.1 M 2-(N-morpholino)ethanesulfonic acid (MES) solution to a final concentration of 30 μM at pH 6.5. Deposit probe spots on the glass slide to produce a microarray. In this embodiment, the spots have a diameter of about 180 μm, with a distance of 300 μm between them. In the investigation on signal variation, 50 repeated TAL1 probe spots are deposited. Thirty repeated spots are used for the 80-mer shuttle hybridization of this invention and for the conventional 80-mer saturated-target hybridization with flat glass format, respectively. For conventional excess-probe hybridization with flat glass format, 300 probe spots are used. For 20-mer single base discrimination studies, 25 spots for each probe are used. For all the microarray used in this experiment, perfect match (PM) and mismatch (MM) probes are deposited on adjacent areas when needed so that the hybridization is performed simultaneously. Following spotting, the slides are incubated in a descicator (˜20% relative humidity) at 25° C. for 18 h. Ultraviolet irradiation are then used to crosslink the oligonucleotides of the 80-mer oligonucleotide probes onto the slide. For 20-mer 5′NH₂-oligonucleotide probes, slides are washed once with 0.1% SDS, twice with deionized water, and then incubated for 5 min with sodium borohydride (NaBH4) solution (in which 2.5 mg of NaBH4 is dissolved in 750 μl of PBS and 250 μl of 100% ethanol); they are then incubated for 3 min with deionized water. The array is then blocked using 5×SSC, 0.1% SDS and 0.1% BSA at 42° C. for 30 min, and rinsed three times in deionized water for 5 min before hybridization.

For 80 mer hybridization, Biotin-labeled target ssDNA is diluted in 50% formamide, 5×SSC and 0.1% SDS, to a final concentration from 0.02 pM to 90 nM. For 20-mer single base discrimination studies, the target concentration is 90 nM. For shuttle hybridization, 1 μl DNA target solution is introduced into the microchannel opening 14 using a pipette. A syringe is then connected to the opening 13, sealing the microchannel. The sample solution is pushed to the distal side of the channel, away from the opening 13, when the syringe is compressed. The distal terminal 14 of the channel is sealed and the compressed air is stored in the distal chamber 14. Hence the sample solution bounces back when the compressed syringe is released.

In shuttle hybridization, the target solution is mixed with the probes 22 while being pumped back and forth inside the entire channel 12, sweeping over the probes 22. The cycle time of the shuttling is 2 seconds.

In static microchannel hybridization, the sample is introduced into the microchannel 12 according to the same scheme but no shuttling is applied. The entire microarray/microchannel assembly is placed in a 42° C. water bath to control the temperature. Following hybridization, the target solution is drawn from the channel 12 and discarded. Several buffer solutions are introduced into, and then drawn out from, the microchannel 12 to perform washing. Each wash takes 5 min. The array is then incubated with 1 μl Cy5-conjugated Streptavidin (SA-Cy5, 0.05 mg/ml) (Zymed, CA) with reagent shuttling for 5 min at 25° C., followed by washing. The microarray 20 is then detached from the microtrench plate 10 to be scanned to detect fluorescence signals.

Conventional flat glass hybridization is conducted as follows. A Gene Frame (Abgene, UK) is attached to the microarray slide to produce a sealed chamber. 300 μl target DNA is used in saturated target hybridization. In excess-probe experiment, 300 repeated TAL1 probe spots are hybridized with 30 μl target DNA. The temperature of hybridization is 42° C., and the slide is incubated in a humidified chamber. Following hybridization, the slides are washed. For 20-mer single base discrimination studies, the target concentration is 90 nM and the volume is 25 μl.

The microarray slides are scanned at a resolution of 5 μm using a GenePix 4000B array scanner (Axon Instrument, CA). The hybridization signals from multiple copies of probes are averaged for analyses.

Experimental Results

In the present invention, the sample solution is shuttled to scan all the probes. It is thus not necessary to fill the microchannel with the sample solution in order to conduct the hybridization. The invented shuttle hybridization method resulted in the fast reaching of equilibrium when the 80-mer target DNA was used. A hybridization time of 500 seconds sufficed to drive the reaction to equilibrium. Rigid PMMA was used as the microfluidic chip substrate to facilitate manual alignment between the microfluidic chip and the microarray chip.

Discrete sample plugs may be generated using air, gas or other fluids to separate the sample plugs in the microchannel. Use the discrete sample plugs in DNA microchannel hybridization to take advantage of the chaotic mixing of droplets to further reduce the reaction time. A 1 μl target may be used for hybridization with an array that holds 5000 probes.

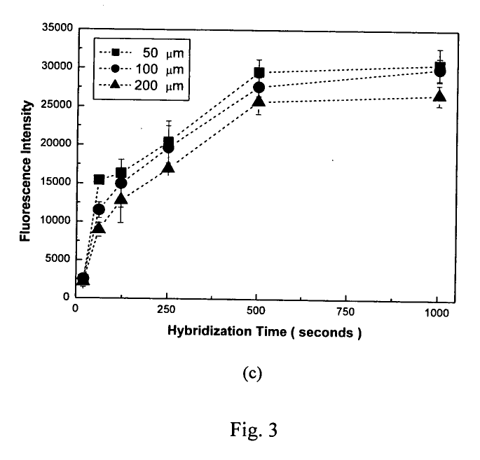

In the present invention, the shuttle hybridization signal reaches equilibrium very rapidly. FIG. 3(a) shows the signal rise for various hybridization formats. As the signal associated with the excess-probe and saturated-target flat glass hybridizations continue to grow beyond four hours, the signal for the invented shuttle hybridization reaches 95% of the final intensity in less than 10 minutes. Notably, the same target concentration is used for all three formats. The final intensity is higher in the saturated-target flat glass hybridization because the target quantity used in the flat glass format is larger.

FIG. 3(b) presents details of the signal growth for a short hybridization time. The signal associated with the invented shuttle hybridization reaches equilibrium more rapidly than the other formats do.

FIG. 3(c) presents the signal growth in shuttle hybridization with various channel depths. The finding reveals that the signals all reach equilibrium after about 500 seconds of hybridization, independent of the channel depth. The small channel dimensions do not explain the short time required to reach equilibrium. The microfluidic droplet chaotic mixing used in this invention reduced the mixing time by two orders of magnitude (64,800 sec/500 sec) below that obtained using conventional flat glass hybridization.

The short equilibrium time implies that the invented microarray biochemical reaction device may be used for DNA quantitative applications directly.

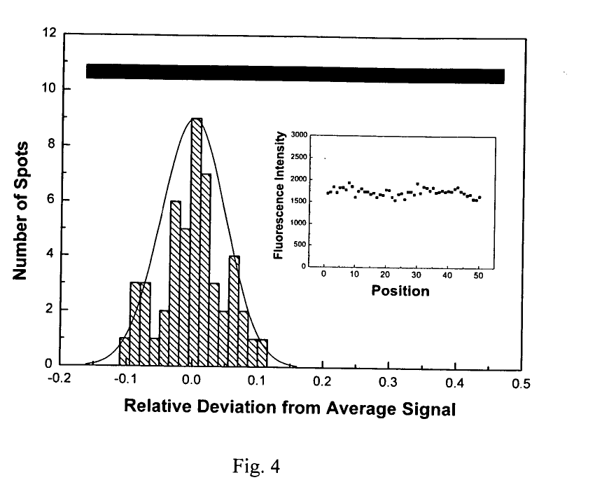

FIG. 4 shows a histogram of the resultant fluorescence signal distribution obtained from hybridization using the present invention. The signal variation associated with the 50 spots is 5.2% (CV). For comparison, the signal variation associated with flat glass hybridization, which involves pure diffusion, is ˜25%. The results herein reveal that 1 μl target solution suffices for a 5000-probe microarray. Moreover, the distance from the first to the 50th straight section is about 1 m. The result demonstrates that the shuttle hybridization offers uniform hybridization efficiency when a small volume is passed through the long channel, as shown in the inset in FIG. 4. No difference between the sections near the sample inlet hole and those near the air chamber is observed.

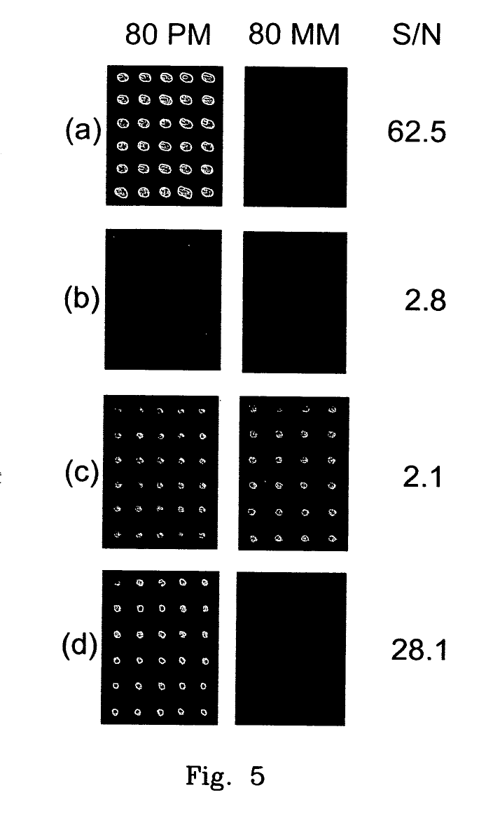

FIG. 5 presents the fluorescence images and the S/N ratio of the perfect match probes (80 PM) and the non-complementary probes (80 MM) hybridized with a perfect match target, for various hybridization formats; wherein (a) denotes to shuttle hybridization at 500 seconds, (b) to static microfluidic hybridization at 500 seconds, (c) to flat glass hybridization at 500 seconds, and (d) to flat glass hybridization at 18 hours. The shuttle hybridization at 500 seconds reveals a highest S/N ratio that is about 2.2 times higher than that obtained by flat glass hybridization time after 18 h.

As the present invention has been shown and described with reference to preferred embodiments thereof, those skilled in the art will recognize that the above and other changes may be made therein without departing form the spirit and scope of the invention.

What is claimed is:

1. A microarray biochemical reaction device, comprising a microarray, on which a plurality of probes may be positioned; a microchannel; and a microfluid driving device; wherein said microarray and said microchannel are so combined that microfluid inside said microchannel may be driven by said microfluid driving device to pass through all probe spots in said microarray; characterized in that said microfluid driving device drives said microfluid to flow forward and backward in said microchannel so that said microfluid scans through probe spots in said microarray repeatedly.

2. The microarray biochemical reaction device according to claim 1, wherein said microchannel is formed in a substrate and said substrate is made of at least one material selected from the group consisted of: plastics, resin, glass and ceramics.

3. The microarray biochemical reaction device according to claim 2, wherein material of said substrate comprises PMMA.

4. The microarray biochemical reaction device according to claim 1, wherein said microchannel comprises a serpentine microtrench.

5. The microarray biochemical reaction device according to claim 1, wherein terminate of said microchannel comprises an air chamber.

6. The microarray biochemical reaction device according to claim 1, wherein said microarray comprises a substrate and probe spots provided in said substrate.

7. The microarray biochemical reaction device according to claim 6, wherein material of substrate of said microarray comprises at least one selected from the group consisted of: glass, plastics, resin and ceramics.

8. The microarray biochemical reaction device according to claim 1, wherein material of substrate of said microarray comprises at least one selected from the group consisted of: soda-lime glass, Pyrex glass, Borofloat glass and, quartz.

9. The microarray biochemical reaction device according to claim 1, wherein said microfluid driving device introduces a gas into said microchannel to separate microfluid in said microchannel to generate discrete fluid plugs.

10. A method for preparation of microarray biochemical reaction device, comprising the following steps:
forming a microtrench in a first substrate;
forming a microarray comprising a plurality of probe spots in a second substrate;
connecting said first substrate and said second substrate, such that microtrench and said microarray form a sealed microfluid channel to allow microfluid in said microfluid channel to pass through all probe spots in said microarray; and
connecting said assembly to a microfluid driving device;
characterized in that said microfluid driving device drives said microfluid to move forward and backward in said microfluid channel to scan all probe spots in said microarray repeatedly.

11. The method according to claim 10, wherein said microtrench is formed by dry etching said first substrate.

12. The method according to claim 11, wherein said microtrench is formed by a laser scriber in a PMMA substrate.

13. The method according to claim 10, wherein said microtrench is formed by wet etching said first substrate.

14. The method according to claim 10, wherein material of said first substrate comprises at least one material selected from the group consisted of: plastics, resin, glass and ceramics.

15. The method according to claim 14, wherein material of said first substrate comprises PMMA.

16. The method according to claim 10, wherein said microchannel comprises a serpentine microtrench.

17. The method according to claim 10, wherein terminate of said microchannel comprises an air chamber.

18. The method according to claim 10, wherein material of said second substrate comprises at least one selected from the group consisted of: glass, plastics, resin and ceramics.

19. The method according to claim 10, wherein material of said second substrate comprises at least one selected from the group consisted of: soda-lime glass, Pyrex glass, Borofloat glass and quartz.

20. The method according to claim 10, wherein said microfluid driving device introduces a gas into said microchannel to separate microfluid in said microchannel to generate discrete fluid plugs.
